(19) United States
(12) Patent Application Publication
(10) Pub. No.: US 2010/0048732 A1
(43) Pub. Date: Feb. 25, 2010

(54) Method of mediating Airway Smooth Muscle Construction Due to Airway Irritation

(76) Inventor: Charles W. Emala, SR., Woodcliff lake, NJ (US)

Correspondence Address: BRYAN CAVE LLP, 1290 AVENUE OF THE AMERICAS, NEW YORK, NY, 10104, US

(21) Appl. No.: 12/227,737

(22) PCT Filed: May 30, 2007
(86) PCT No.: PCT/US2007/012848

§ 371 Date: Sep. 18, 2009

Related U.S. Application Data
(60) Provisional application No. 60/809,276, filed on May 30, 2006.

(51) Int. Cl. A61K 31/05; A61M 16/12

(52) U.S. Cl. 514/731; 128/203.12

(57) ABSTRACT

Methods of minimizing bronchospasm and contraction of airway smooth muscle due to irritation of the airway are provided. More particularly, a method is provided of mitigating bronchospasm or airway smooth muscle constriction due to irritation. This method includes administering to a subject in need of such treatment an amount of propofol or a derivative thereof effective to decrease the severity and/or duration of bronchospasm or airway smooth muscle constriction. Also provided are methods of up-regulating GABA mediated relaxation of airway smooth muscle at GABA_{A} receptors expressed on airway smooth muscle and methods of anesthetizing a subject and minimizing bronchospasm or airway smooth muscle constriction due to irritation using propofol or a propofol derivative.

### FIELD OF THE INVENTION

The present invention relates to methods of minimizing bronchospasm and contraction of airway smooth muscle due to irritation of the airway. More particularly, the present invention relates to a method of mitigating bronchospasm or airway smooth muscle constriction due to irritation including administering to a subject in need of such treatment an amount of propofol or a derivative thereof effective to decrease the severity and/or duration of bronchospasm or airway smooth muscle constriction.

### BACKGROUND OF THE INVENTION

The incidence of asthma is increasing worldwide with a 250% increase in the United States over the past 20 years. In 2001, the National Institutes of Health estimated that 17 million Americans suffer from asthma (1) and 12.1 million from Chronic Obstructive Pulmonary Disease (COPD) (2). An increasing number of patients with these diseases require anesthesia and bronchospasm especially during induction and emergence from anesthesia carries significant morbidity. Methods to minimize bronchospasm during anesthesia will make anesthetic care safer for a growing number of patients with asthma and COPD.

Intubation of the trachea during induction or the presence of an endotracheal tube during emergence from anesthesia initiates a neurally-mediated irritant reflex in the airway promoting bronchoconstriction. Neural control of airway tone is modulated by both cholinergic nerves traveling within the vagus nerve and by nocioceptive C fibers that send afferent signals to the CNS that modulate cholinergic outflow and locally release tachykinins into the airway wall. In brain, tachykinins release γ-amino butyric acid (GABA), the primary neuronal inhibitory neurotransmitter. The cholinergic component of this reflex has been extensively explored in animal models and humans but little is known regarding the contribution of C fibers, released tachykinins, or GABA to reflex-induced bronchoconstriction.

Propofol is known to allosterically enhance the activity of GABA at GABA_{A} receptors in the brain and is recognized as the intravenous anesthetic induction agent of choice in patients at risk for bronchospasm (3, 4) but its mechanism of airway protection is poorly understood. Previous studies of propofol's effect on cholinergic outflow or airway smooth muscle have not adequately accounted for the mechanism of propofol's protective airway effects. Elucidating the mechanisms of propofol's protective airway effects may provide novel therapies for bronchoconstriction from many causes.

The mechanism of this airway protection has largely been attributed to attenuation of airway neural reflexes because studies on the direct effects of propofol on airway smooth muscle have suggested that supra-clinical concentrations of propofol are necessary to block muscarinic mediated responses. These studies have made two important and perhaps incorrect assumptions: that cholinergic nerves are the primary nerves activated during reflex induced bronchoconstriction and that the primary contractile agonist acting at the airway smooth muscle is acetylcholine.

Propofol's preferential protection from reflex-induced bronchoconstriction during intubation has traditionally been attributed to its action on airway cholinergic nerves and less convincingly to modulation of L-type calcium channels (5) or inositol phosphate signaling (6) in airway smooth muscle. These previous studies have shown these smooth muscle effects only at concentrations of propofol above those achieved clinically. (>100 μM). (18-20.) However, propofol is not better at attenuating airway constriction by either cholinergic nerve activation or by direct activation of smooth muscle muscarinic receptors compared to other intravenous induction agents (e.g. thiopental).

Many studies in airway tissues from humans and animal models have shown that propofol can attenuate contractile responses from acetylcholine (7-9), histamine (10), and endothelin (9), but only at concentrations of 100-300 μM. Muscarinic receptor-mediated signaling coupled to L-type calcium channels (5), intracellular calcium changes (10, 11) or inositol phosphate synthesis (6) have only been effected by these high concentrations of propofol (>100 μM). Calcium sensitivity in permeabilized canine tracheal smooth muscle cells in the absence or presence of muscarinic receptor activation was not effected even at concentrations of 270 μM (12). In dogs, propofol attenuated methacholine bronchoconstriction (13), the neural component of histamine-induced bronchoconstriction (9) or vagal nerve-induced bronchoconstriction (14) but only at concentrations of 20 mg/ml propofol (typical human induction dose is 2-3 mg/kg). Taken together, these studies have demonstrated that at clinically relevant concentrations of propofol, propofol does not have significant effects on cholinergic modulation of airway smooth muscle.

Conversely, in a sheep model, vagal nerve mediated bronchoconstriction has been shown to be more sensitive to low concentrations of propofol than cholinergic constriction mediated at the airway smooth muscle. Delivery of propofol via the bronchial artery to sheep resulted in attenuation of vagal nerve induced bronchospasm at lower doses (0.3 mg/min) and attenuation of methacholine induced bronchoconstriction only at doses (3 mg/min) believed to be above clinically relevant concentrations by these authors (15, 16).

It is well established that the primary site of anesthetic action of propofol, etomidate, and thiopental is by allosteric potentiation of the action of γ-amino butyric acid (GABA) on GABA_{A} receptors in the CNS resulting in inward inhibitory chloride currents (26-28). GABA is the primary inhibitory neurotransmitter in the central nervous system. GABA receptors consist of both ionotropic (GABA_{A} and GABA_{C}) and metabotropic (GABA_{B}) receptors (29). The GABA_{A} and GABA_{C} receptors are pentameric ligand gated ion channels that conduct chloride currents resulting in hyperpolarization of the cell membrane impeding the effect of depolarizing (i.e. stimulatory) signals (30, 31). Hyperpolarization of an airway smooth muscle cell is important in attenuating inward flux of calcium through voltage-dependent calcium channels (32-34). GABA_{A} receptors are classically pentamers composed of combinations of subunit subtypes (α₁₋₆, β₁₋₃, γ₁₋₃, δ, ε, π, θ) which dictate pharmacologic and gating properties of this chloride channel in the mammalian brain (29, 35, 36). This diversity is further extended by splice variants for multiple subunits (37, 38). Despite the enormous combinational possibilities most GABA_{A} receptors in the CNS are composed of α, β, and γ subunits in a 2:2:1 ratio (39, 40). In addition to the orthosteric binding site for GABA, subunit composition dictates mechanisms of channel regulation by a wide variety of allosteric binding sites for anesthetics, benzodiazepines, convulsants, and neurosteroids (41).

GABA receptors are ubiquitously expressed in the central nervous system and the modulation of neuronal activity by GABA has been extensively studied. GABA receptors are also expressed in the peripheral nervous system where they also serve an inhibitory function (42, 43). In contrast, the expression of GABA receptors in non-neuronal cells has received limited study. Initial attempts to survey the expression of GABA receptors outside the central nervous system relied upon RT-PCR analysis of RNA isolated from whole peripheral organs (44-47). Although these studies suggested ubiquitous expression of many GABA receptor subunits it is unknown what cellular components of these tissues were expressing GABA receptor subunits. It is possible that these studies identified GABA receptor subunits expressed in peripheral nerves contained within these organs. More specific expression of GABA receptor subunits were identified in neuroendocrine cells including pancreatic beta (48, 49), pituitary (50) and adrenal cells (51). Indirect pharmacologic evidence suggested GABA receptor expression in vascular (52), bladder (53), uterine (45, 54, 55) and gut (56) smooth muscle in addition to the identification of GABA receptors in the peripheral nerves that innervate these tissues (57-60). The expression of GABA receptors in these smooth muscles of gut, bladder, vascular, and uterine smooth muscle was inferred from pharmacologic responses as opposed to a direct molecular identification of GABA receptors within the smooth muscle (45, 52-56, 61). Subsequently, subunits of GABA receptors have been demonstrated in heart (62), uterus (63, 64), kidney (65), liver (66, 67) and fibroblasts (68).

GABA receptors have been identified on nerves in the lung and have been shown to modulate cholinergic outflow to the lung both in the brainstem (69, 70) and in the periphery. Conversely, it is believed that GABA receptors have never been described in airway smooth muscle itself. It has been known for some time that GABA_{B}-specific agents decrease electrically field-stimulated airway constriction by modulating acetylcholine release from parasympathetic nerves (71-75). This is mediated by a pre-synaptic inhibition of acetylcholine release by GABA_{B} receptors. Previous studies have discounted a role for GABA receptors in the direct modulation of airway smooth muscle function but these studies have typically been performed with GABA rather than a GABA_{A}-selective agonist (72-74). Simultaneous activation of smooth muscle GABA_{A} and GABA_{B} receptors may not allow for relaxation effects of GABA_{A} receptors to be elucidated. Two previous studies failed to show an effect of the GABA_{A}-selective agonist muscimol on basal tone (73, 74) or acetylcholine-induced contraction (74) in guinea pig airway rings. However, these studies were limited to a single contractile agonist (acetylcholine) and more importantly did not address the potential ability of muscimol to relax contracted tissue as opposed to muscimol's ability to impair an initial contraction.

Airway afferent nerves that would be activated by an irritant such as an endofracheal tube have been subclassified by multiple characteristics including location within the airway, physiochemical sensitivity, neurochemistry, and conduction velocities (82-85). Three broad groups of airway afferent nerves are 1) unmyelinated nocioceptive C fibers, 2) rapidly adapting or irritant mechanoreceptors (RARs), and 3) slowly adapting stretch receptors (SARs). Stimuli that activate RARs or nocioceptive C fibers induce reflex bronchonconstriction in animals and humans (86-90). Nocioceptive C fibers, in addition to sending an afferent signal to the CNS to modulate cholinergic outflow, also locally liberate tachykipins into the airway which have many airway effects.

Tachykinins have been known for many years to have a myriad of effects in airways including bronchoconstriction, hyperemia, microvascular hyperpemeability, and mucus secretion via effects on airway smooth muscle, mucosal vasculature, and submucosal glands and mast cells. Direct effects of tachykinins on airway smooth muscle has been demonstrated in multiple species including man, acting upon all three known subtypes of neurokinin receptors (NK1, NK2, and NK3) (91-93). Interest in the role of tachykinins in patients with asthma (94) was initially stimulated by the findings that a tachykinin antagonist FK224 attenuated bradykinin induced bronchoconstriction in asthmatics (95). More recently, interest in the role of tachykinins in asthma and COPD (96, 97) and the demonstration of a relationship between reactivity to methacholine and tachykinins in asthmatic airways (98) has led to studies demonstrating the effectiveness of dual (99) or triple (100) neurokinin subtype-specific antagonists in blocking neurokinin induced bronchoconstriction in asthmatics. Despite these important documented roles of tachykinins in bronchoconstriction and more specifically in reflex-induced bronchoconstriction, nothing is known about the interaction of intravenous anesthetics with tachykinins during a routine irritant to the upper airway: an endotracheal tube.

In view of the foregoing, it would be advantageous to provide a method to mediate bronchospasm and airway smooth muscle contraction due to irritation of the airway by activation of GABA_{A} receptors in airway smooth muscle.

### SUMMARY OF THE INVENTION

One embodiment of the present invention is a method of mitigating bronchospasm or airway smooth muscle constriction due to irritation. This method includes administering to a subject in need of such treatment an amount of propofol or a derivative thereof effective to decrease the severity and/or duration of bronchospasm or airway smooth muscle constriction.

Another embodiment of the present invention is a method of up-regulating GABA mediated relaxation of airway smooth muscle at GABA_{A} receptors expressed on airway smooth muscle. This method includes administering to a subject an amount of propofol or a propofol derivative effective to increase the speed of the spontaneous relaxation of the airway smooth muscle.

A further embodiment of the present invention is a method of anesthetizing a subject and minimizing bronchospasm or airway smooth muscle constriction due to irritation. This method includes administering to a subject in need of such treatment an effective amount of propofol or a derivative thereof directly to the airway smooth muscle concurrently with anesthetization.

### BRIEF DESCRIPTION OF THE DRAWINGS

FIG. 1 shows a comparison of the ability of intravenous anesthetics to relax airway smooth muscle contractions in isolated rings of guinea pig trachea in response to acetylcholine.

FIG. 2 shows a comparison of the ability of propofol or thiopental in attenuating airway contractions induced by either neurally liberated tachykinins (NANC contractions) or exogenous tachykinins (substance P) in epithelium-denuded guinea pig tracheal rings.

FIG. 3 shows the ability of etomidate or ketamine to relax contractions induced by cholinergic nerve stimulation, C fiber stimulation, or exogenous tachykinins in guinea pig tracheal rings.

FIG. 4 shows the effects of clinically relevant concentrations of propofol or thiopental on relaxation of histamine or endothelin-1 induced contractions.

FIG. 5 shows the effects of cumulative concentrations of propofol or thiopental on contracted human tracheal rings suspended in organ baths with substance P.

FIG. 6 shows a comparison of propofol relaxation on NK2- and NK1-mediated contraction in guinea pig tracheal rings.

FIG. 7 shows expression of mRNA encoding NK1, NK2, and NK3 receptors in native airway smooth muscle from guinea pig and human and in cultured human airway smooth muscle cells.

FIG. 8 shows dose-dependent relaxation of substance P contraction guinea pig tracheal rings by the GABA_{A} agonist muscimol.

FIG. 9 shows dose-dependent relaxation of substance P contraction in human tracheal rings by the GABA_{A} agonist muscimol.

FIG. 10 shows the ability of gabazine (a GABA_{A} antagonist) to reverse propofol mediated relaxation of a substance P contraction.

FIG. 11 shows mRNA expression of subunits of GABA_{A} receptors in airway smooth muscle cells from native guinea pig and human airway smooth muscle and cultured human airway smooth muscle cells.

FIG. 12 shows immunoblot analysis identifying selected GABA_{A} subunits in native guinea pig and human airway smooth muscle and cultured human airway smooth muscle cells.

FIG. 13 shows expression of a β subunit of GABA_{A} receptors is immunohistochemically localized to airway smooth muscle in guinea pig tracheal rings.

FIG. 14 shows spontaneous relaxation of an NK2 mediated contraction and NK1-mediated contraction in guinea pig tracheal rings.

FIG. 15 shows spontaneous relaxation of NK2-induced contraction of guinea pig tracheal rings in the presence of an NK2 agonist.

FIG. 16 shows the effect of pretreatment of guinea pig tracheal rings with an inhibitor of GABA re-uptake on propofol-mediated relaxation of a substance P-induced contraction.

FIG. 17 shows immunohistochemical detection of abundant amounts of GABA in an area immediately adjacent to airway smooth muscle in guinea pig tracheal ring.

FIG. 18 shows a representative chromatogram of amino acid neurotransmitters (including GABA) from a tissue lysate.

FIG. 19 shows a representative study of airway pressure and hemodynamic measurements in an intact guinea pig treated with repetitive intravenous challenges of capsaicin resulting in similar changes in airway pressure.

FIG. 20 shows GABA-induced chloride current in airway smooth muscle cells.

FIG. 21 shows current traces of GABA concentration dose response in an HEK 293 cell transiently transfected and expressing the ρ1 GABAc subunit.

### DETAILED DESCRIPTION OF THE INVENTION

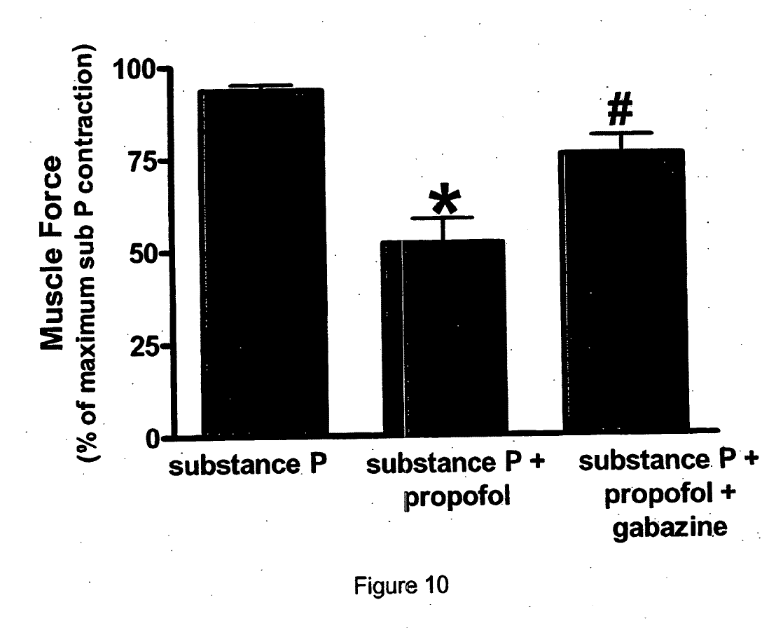

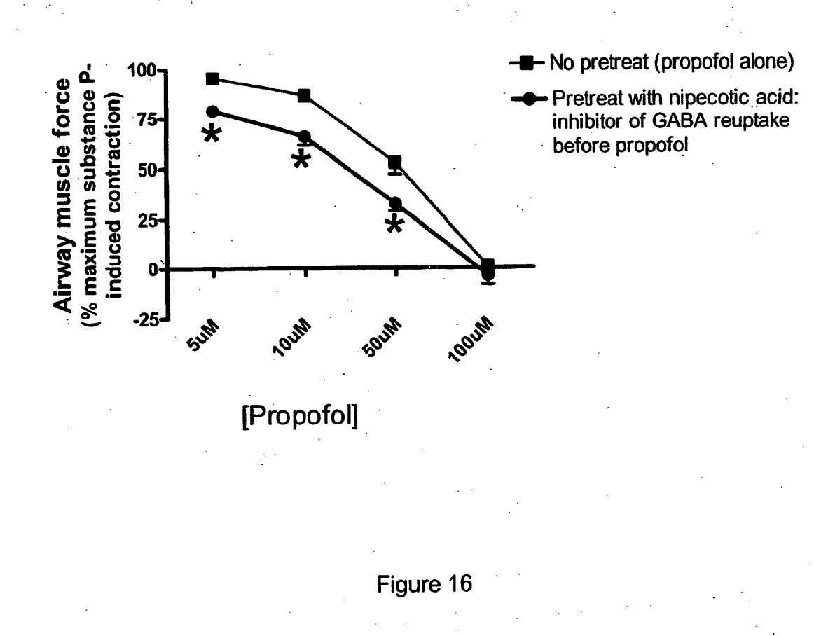

Surprisingly, our studies suggest that propofol potentiates GABA mediated relaxation at GABA_{A} receptors expressed on airway smooth muscle. (FIGS. 10 and 16.) However, clinically relevant concentrations of etomidate were without effect. This at first seems in conflict since the anesthetic effect of propofol and etomidate are mediated by GABA_{A} receptors. However, clinically achieved concentrations of etomidate are considerably lower than propofol concentrations. Perhaps more importantly, the subunit composition of GABA_{A} channels is known to effect their sensitivity for propofol versus etomidate. While the inclusion of a β2 or β3 subunit appears crucial for etomidate's activity at GABA_{A} receptors (76-78), alterations in the β subunit structure have pronounced effects on etomidate but not propofol's functional effects (79,80). The inclusion of other subunits such as the θ subunit into the pentameric GABA_{A} receptor confer differential sensitivity to propofol versus etomidate (81). Thus it would appear that the subunit composition of the GABA_{A} subunits expressed in airway smooth muscle will be more sensitive to propofol than etomidate.

A novel hypothesis for the pathophysiology of some forms of asthma has suggested that overactivity of airway nocioceptive C fibers is analogous to “hyperreflexia” seen in cutaneous pain syndromes which are also modulated by C fibers. Endogenous tachykinins released in peripheral tissues from C fibers may play a role in symptoms of hyperalgesia and allodynia (101-103). Inflammatory responses in lung are known to increase tachykinin expression and contribute to “hyperreflexia” of airway large-diameter (A-fiber) nodose ganglion neurons (104). Teleologically, similarities between tachykinin-mediated skin and lung hyperreflexia may not be surprising since both communicate with the external environment.

Tachykinin receptors are known to be expressed on airway nerves (105-108) and the release of GABA by activation of tachykinins is not without precedent. Tachykinins acting at different neurokinin receptor subtypes have been shown to liberate GABA in the ventrolateral preoptic area of rats (109), the entorbinal cortex of rats (110), the mouse striatum (111), and rat spinal cord (112, 113). Accordingly, it appears that tachykinins, specifically those activating NK2 receptors, may liberate GABA from airway nerves.

A previously uncharacterized mechanism by which certain members of this class facilitate airway smooth muscle relaxation is described herein. Although GABA_{A} receptors are a well recognized target of intravenous anesthetics in the central nervous system, the expression of GABA receptors on airway smooth muscle and their modulation by intravenous anesthetics, it is believed, has never been described. Surprisingly, our data demonstrate for the first time that 1) GABA is locally present near airway smooth muscle, 2) airway smooth muscle expresses GABA_{A} receptors, 3) GABA_{A} agonists relax airway smooth muscle, and 4) propofol selectively attenuates NK2-mediated airway constriction via GABA_{A} receptors.

Accordingly, one embodiment of the present invention is a method of mitigating bronchospasm or airway smooth muscle constriction due to irritation. This method includes administering to a subject in need of such treatment an amount of propofol (2,6-diisopropylphenol) or a derivative thereof effective to decrease the severity and/or duration of bronchospasm or airway smooth muscle constriction.

Another embodiment of the present invention is a method of up-regulating GABA mediated relaxation of airway smooth muscle at GABA_{A} receptors expressed on airway smooth muscle. This method includes administering to a subject an amount of propofol or a propofol derivative effective to increase the speed of the spontaneous relaxation of the airway smooth muscle.

A further embodiment of the present invention is a method of anesthetizing a subject and minimizing bronchospasm or airway smooth muscle constriction in the subject due to irritation. This method includes administering to a subject in need of such treatment an effective amount of propofol or a derivative thereof directly to the airway smooth muscle concurrently with anesthetization.

An “effective amount” is an amount sufficient to effect beneficial or desired clinical results. An effective amount can be administered in one or more doses. In terms of treatment, an “effective amount” of propofol or a derivative thereof is an amount sufficient to palliate, ameliorate, stabilize, reverse, slow or delay a bronchospasm or airway smooth muscle constriction or otherwise up-regulate GABA mediated relaxation of airway smooth muscle at GABA_{A} receptors expressed on airway smooth muscle. Detection and measurement of these indicators of efficacy are discussed below. The effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the drug being administered. For instance, the concentration of a propofol derivative need not be as high as that of propofol itself in order to be therapeutically effective.

An effective amount of propofol is typically up to about 15% (wt) propofol, such as for example, up to about 10% (wt) propofol, including, for example, about 1%, 2%, 3%, 4%, 5%, 6%, 7%, 8%, and 9% propofol.

Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size and species of animal, and like factors well known in the arts of medicine and veterinary medicine. In general, a suitable dose of propofol or a propofol derivative will be that amount of the compound which is the lowest dose effective to produce the desired effect. The effective dose of propofol or a propofol derivative maybe administered as two, three, four, five, six or more sub-doses, administered separately at appropriate intervals throughout the day.

Propofol or a propofol derivative may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, propofol or a propofol derivative may be administered in conjunction with other treatments. Propofol or a propofol derivative maybe encapsulated or otherwise protected against gastric or other secretions, if desired.

While it is possible for a propofol or a propofol derivative of the invention to be administered alone, it is preferable to administer the propofol or propofol derivative as a pharmaceutical formulation (composition). The pharmaceutical compositions of the invention comprise propofol or a propofol derivative as an active ingredient in admixture with one or more pharmaceutically-acceptable carriers and, optionally, one or more other compounds, drugs, ingredients and/or materials. Regardless of the route of administration selected, the propofol or propofol derivatives of the present invention are formulated into pharmaceutically-acceptable dosage forms by conventional methods known to those of skill in the art. See, e.g., Remington's Pharmaceutical Sciences (Mack Publishing Co., Easton, Pa.).

Pharmaceutical carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each carrier used in a pharmaceutical composition of the invention must be “acceptable” in the sense of being compatible with the other ingredients of the formulation and not injurious to the animal. Carriers suitable for a selected dosage form and intended route of administration are well known in the art, and acceptable carriers for a chosen propofol or propofol derivative, dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and soibitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the animal. Ingredients and materials suitable for a selected dosage form and intended route of administration are well known in the art, and acceptable ingredients and materials for a chosen propofol or propofol derivative, dosage form and method of administration can be determined using ordinary skill in the art.

Preferably, for example, the pharmaceutical compositions of the present invention are administered by inhalation. Delivery by inhiation may be accomplished using, e.g., metered-dose inhalers, nebulizers, or micronized dry powders. In a metered-dose inhaler, the pharmaceutical compositions of the present invention are dissolved in a low boiling point liquid in a pressurized canister, and actuation of the inhaler is coordinated with inhalation by the patient to deliver the pharmaceutical compositions of the present invention to the lungs. Nebulizers produce droplets of a liquid formulation containing pharmaceutical compositions of the present invention by passing a stream of gas or oxygen through a reservoir of the liquid formulation. The droplet-containing stream of gas or oxygen is then inhaled by the patient through a facemask or mouthpiece. Alternatively, an ultrasonic nebulizer produces droplets by vibration, which are then inhaled. A dose of a micronized powder containing the pharmaceutical compositions of the present invention may be delivered using a pressurized inhaler with a valve to deliver a metered amount of the powder. Capsules or cartridges, e.g., containing a powder mix of the pharmaceutical compositions of the present invention and a suitable powder base, if desired, may be provided for use in the pressurized inhaler.

Pharmaceutical formulations suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Formulations for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Formulations which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more of propofol or a propofol derivative in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

A “subject” is a vertebrate, preferably a mammal, more preferably a human. Mammals include farm and sport animals, and pets.

As used herein, a propofol derivative is a propofol compound that has been modified in any manner from its original structure (i.e., 2,6-diisopropylphenol) and is able to mitigate bronchospasm, to mitigate airway smooth muscle constriction, or is able to up-regulate GABA mediated relaxation of airway smooth muscle at GABA_{A} receptors expressed on smooth muscle.

Other compounds which demonstrate GABA agonistic activity, include for example, muscimol, progabide, riluzole, baclofen, gabapentin, vigabatrin, valproic acid, tiagabine, lamotrigine, pregabalin, phenytoin, carbamazepine, topiramate, derivatives and prodrugs thereof, and pharmaceutically acceptable salts of these GABA agonists, derivatives, and prodrugs.

The following examples are provided to further illustrate the compositions and methods of the present invention. These examples are illustrative only and are not intended to limit the scope of the invention in any way.

### EXAMPLES

### Example 1

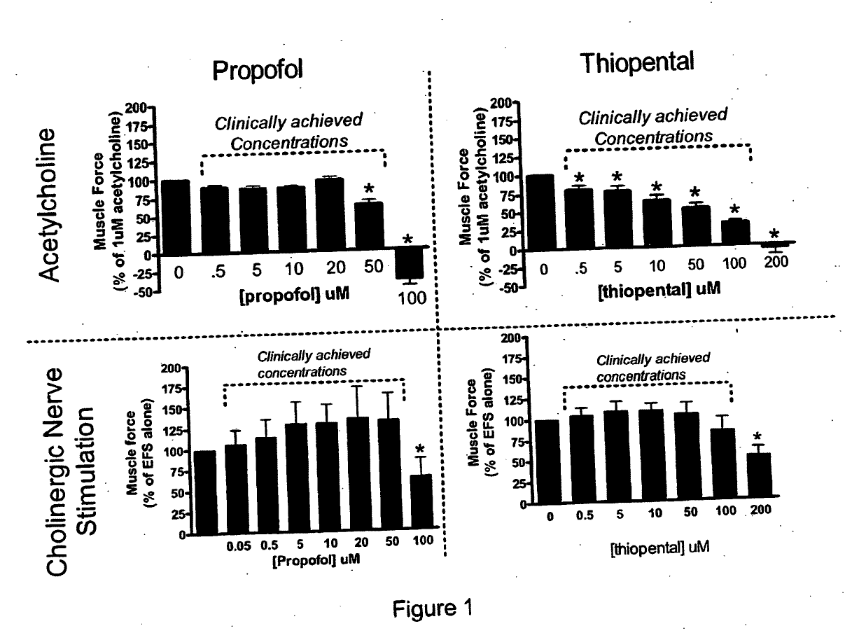

Intravenous anesthetics were compared for their ability to relax airway smooth muscle contractions in isolated rings of guinea pig trachea in response to acetylcholine. As shown in FIG. 1, thiopental, in clinically significant concentrations (114, 115) dose-dependently relaxed acetylcholine-induced contractions while clinically relevant concentrations of propofol (18-20) were without effect.

These results are in conflict with the multiple clinical studies showing that propofol protects better against reflex-induced bronchoconstriction than thiopental (3, 4). These findings raise at least two possibilities; 1) intravenous anesthetic effects at airway smooth muscle are unrelated to their observed protective clinical effects (i.e. the nerve is more important) or 2) an agonist other than acetylcholine is acting upon airway smooth muscle in irritant induced bronchoconstriction.

To distinguish between the two possibilities, intravenous anesthetics were evaluated for their effects on attenuating bronchoconstriction induced by activation of the cholinergic nerves (liberating acetylcholine). If cholinergic nerve activity is the major component of reflex bronchoconstriction, propofol would be expected to be more effective than other intravenous anesthetics at blocking airway contraction. Clinically relevant concentrations of propofol failed to relax cholinergic nerve mediated contractions. (FIG. 1.)

### Example 2

### Clinically Relevant Concentrations of Propofol Relax C Fiber-Induced Contractions Released Tachykinins) or Contractions Induced by Exogenous Tachykinins

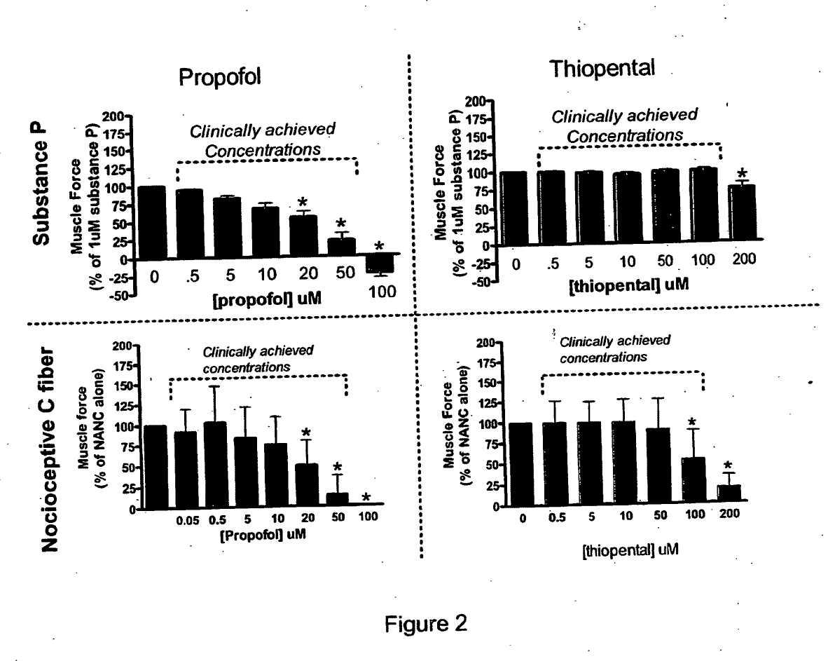

Substance P is an endogenous tachykinin released in airways by C fibers during irritation of the airway. Propofol was more effective than thiopental in attenuating airway contractions induced by either neurally liberated tachykinins (NANC contractions) or exogenous tachykinins (substance P) in epithelium-denuded guinea pig tracheal rings. (FIG. 2.) These results call into question the presumption that reflex induced bronchoconstriction is primarily a cholinergic nerve mediated event and that the key contractile agonist is acetylcholine.

### Example 3

### Clinically Relevant Concentrations of Etomidate or Ketamine do not Relax Contractions Induced by Cholinergic Nerve Stimulation, C Fiber Stimulation, or Exogenous Tachykinins in Guinea Pig Tracheal Rings

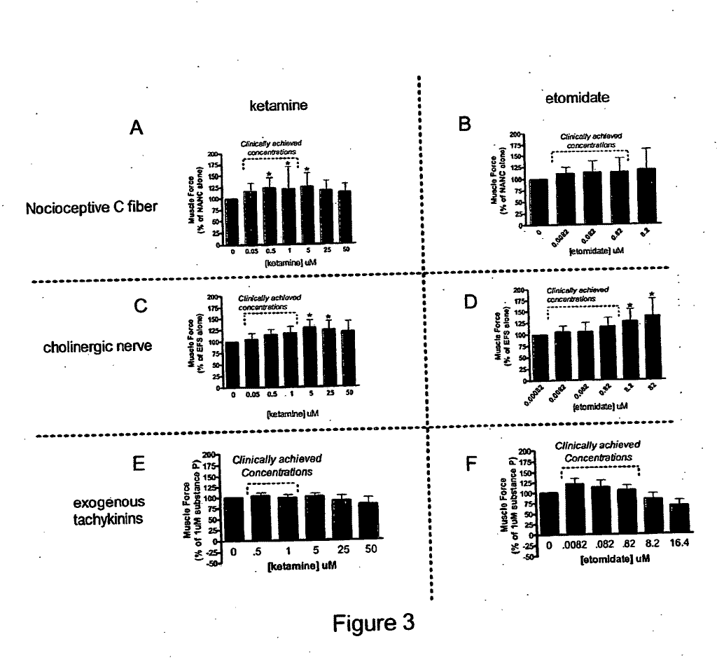

Propofol is known to have preferential protective airway effects during intubation compared to other intravenous anesthetics (3, 4). To determine if two other commonly used intravenous anesthetics have similar protective airway effects on contractile challenges, etomidate and ketamine were evaluated to determine their ability to relax airway smooth muscle contraction induced by NANC nerve stimulation (FIGS. 3A and B), cholinergic nerve stimulation (FIGS. 3C and D), and exogenous addition of substance P (FIGS. 3E and F) to isolated guinea pig tracheal rings in organ baths.

Clinically relevant concentrations of ketamine (116) or etomidate (117) failed to relax airway constriction by any of these contractile agonists. (FIG. 3.) This further highlights the selective ability of clinically relevant concentrations of propofol (18-20) to relax airway smooth muscle and that this relaxation was selective for tachykinins and NANC contractions mediated by tachykinins.

### Example 4

### Clinically Relevant Concentrations of Propofol or Thiopental Fail to Significantly Relax Histamine Or Endothelin-1 Induced Contractions

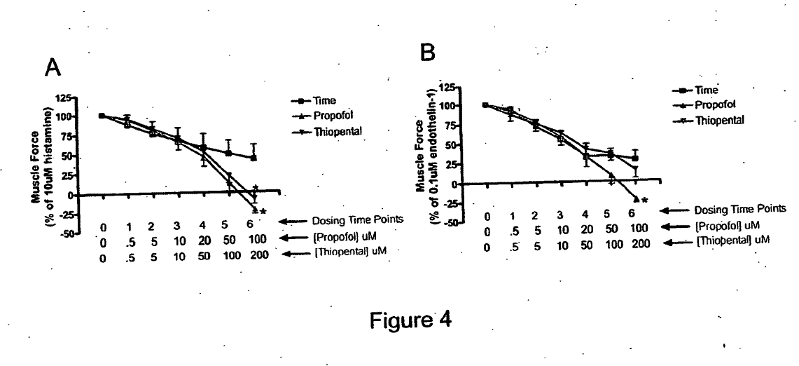

To determine if propofol and thiopental have the ability to relax histamine or endothelial induced contractions, propofol and thiopental were evaluated at clinical and supra-clinical concentrations to determine their ability to relax histamine or endothelin-1 induced contractions in guinea pig tracheal rings. FIG. 4 shows that propofol and thiopental did not differ in their ability to relax either a histamine or endothelin-1 induced contraction. In fact, within clinically relevant concentrations, neither anesthetic effected the spontaneous relaxation of these contractions. Taken together with the acetylcholine, substance P, and nerve stimulation results, these data further support the unique effect of clinically relevant concentrations (18-20) of propofol to mediate tachykinin-induced contractions.

### Example 5

### In Human Tracheal Rings, Tachykinin-Induced Contraction is Relaxed by Clinically Relevant Concentrations of Propofol but not Thiopental

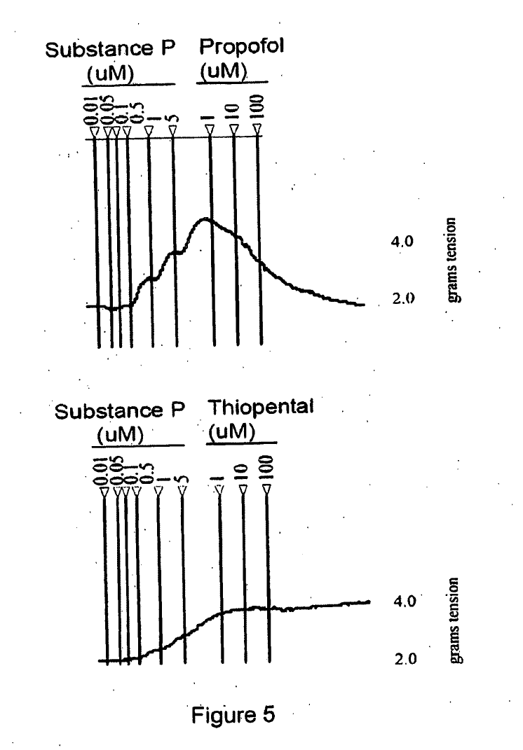

To determine if these results applied to human airway smooth muscle, human tracheal rings suspended in organ baths were contacted with substance P and treated with cumulative concentrations of propofol or thiopental to attempt to relax the muscle. Substance P dose-dependently contracted human tracheal rings, and propofol, but not thiopental, dose-dependently relaxed this contraction within clinically relevant concentrations of propofol (18-20). (FIG. 5.)

This confirms for functional human airway rings, the observation in guinea pig tracheal rings that propofol is more effective than thiopental in relaxing a tachykinin-induced contraction. Moreover, it further challenges the clinical assumption that the benefit of. propofol in patients with reactive airway disease is due to attenuation of a cholinergic/acetylcholine induced contraction. Rather, it suggests that propofol's preferential effects are related to tachykinins.

### Example 6

### Propofol Preferentially Relaxes an NK2-Versus NK1-Mediated Contraction in Guinea Pig Tracheal Rings

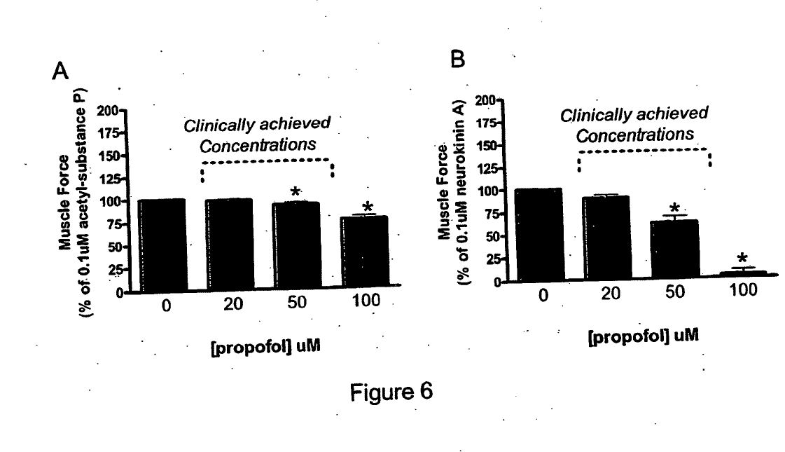

Because both of these subtypes have been reported to be expressed in airway smooth muscle, an experiment to determine whether propofol was more effective against an NK1 or NK2-induced contraction was carried out. Guinea pig tracheal rings stimulated with acetyl-substance P to produce an NK1-induced contraction (FIG. 6A) and with neurokinin A to produce an NK2-induced contraction (FIG. 6B). Propofol significantly relaxed an NK2-induced contraction (FIG. 6B), but was much less effective against an NK1-induced contraction (FIG. 6A).

### Example 7

### Native Airway Smooth Muscle from Guinea Pig and Human, and Cultured Human Airway Smooth Muscle Cells Express mRNA Encoding NK1, NK2, and NK3 Receptors

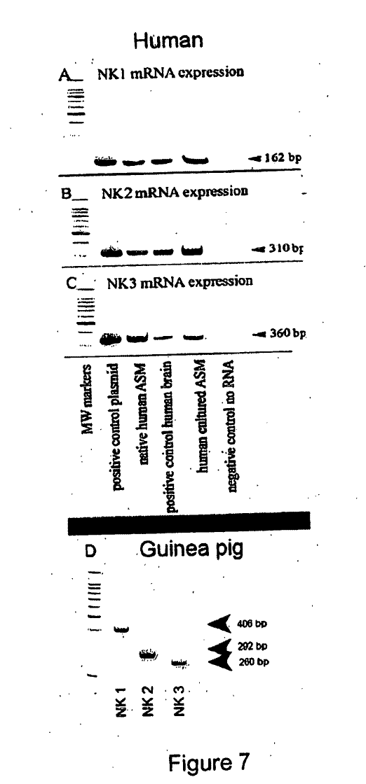

Three subtypes of tachykinin receptors are known. Both native human and guinea pig, as well as, human cultured airway smooth muscle expressed mRNA encoding not only the NK1 and NK2 receptor subtypes, but also the NK3 receptor mRNA. (FIG. 7.)

### Example 8

### An Agonist at the GABA_{A} Receptor, Muscimol, Dose-Dependently Mimics Propofol's Relaxation of a Tachykinin-Induced Contraction in Guinea Pig and Human Tracheal Rings and An Antagonist at the GABA_{A} Receptor Attenuates Propofol-Induced Relaxation

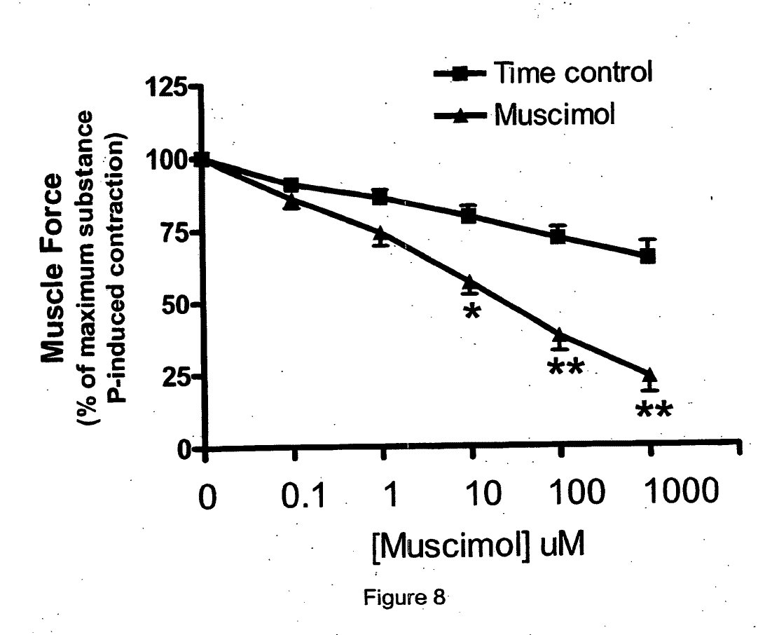

Propofol's effect as an anesthetic is modulated by an allosteric potentiation of GABA at GABA_{A} receptors in the brain. However, we are unaware of any report that GABA_{A} receptors have been found on airway smooth muscle. If propofol was acting via GABA_{A} receptors on airway smooth muscle, then it should be possible to mimic this relaxation of a tachykinin receptor with an agonist of the GABA_{A} receptor. The GABA_{A} agonist muscimol, dose-dependently relaxed a substance P contraction. (FIG. 8.)

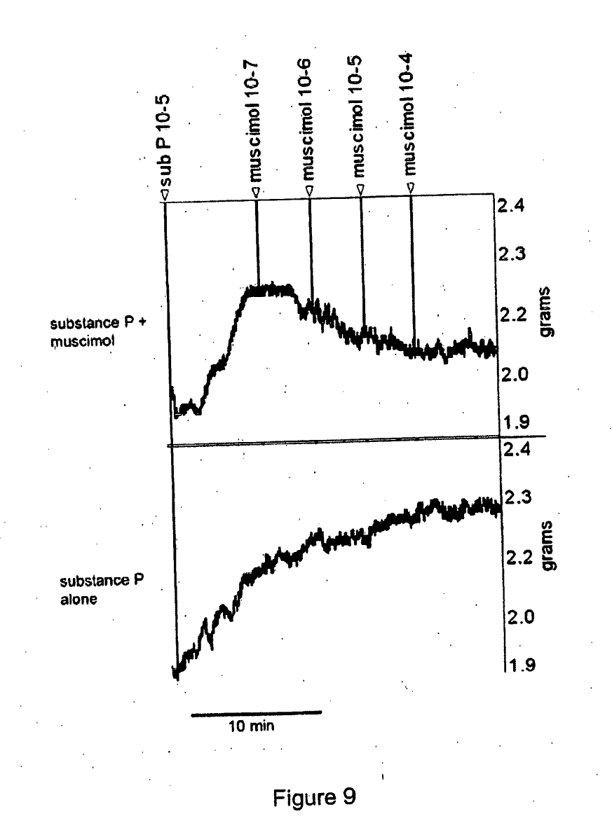

To determine whether this mechanism was feasible in the human airway, smooth muscle human tracheal rings were contracted with substance P. Muscimol dose dependently relaxed this contraction. (FIG. 9.) To determine if the mechanism of propofol's relaxation of a tachykinin response was mediated by GABA_{A} receptors, the ability of an antagonist of GABA_{A} receptors should inhibit propofol's response was evaluated. Gabazine, a GABA_{A} antagonist, was able to reverse propofol mediated relaxation of a substance P contraction. (FIG. 10.)

### Example 9

### The mRNA Encoding Multiple Subunits of GABA_{A} Receptors and GABA_{A} Subunit Proteins are Expressed in Native Guinea Pig and Human Airway Smooth Muscle and in Cultured Human Airway Smooth Muscle Cells

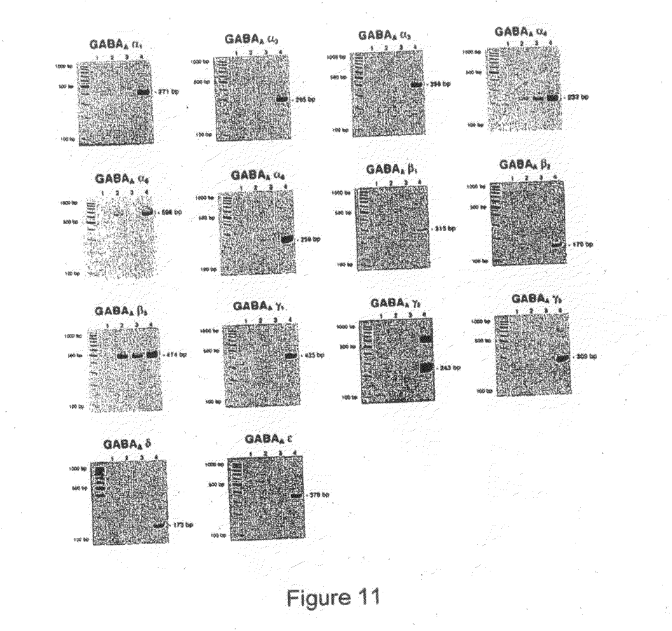

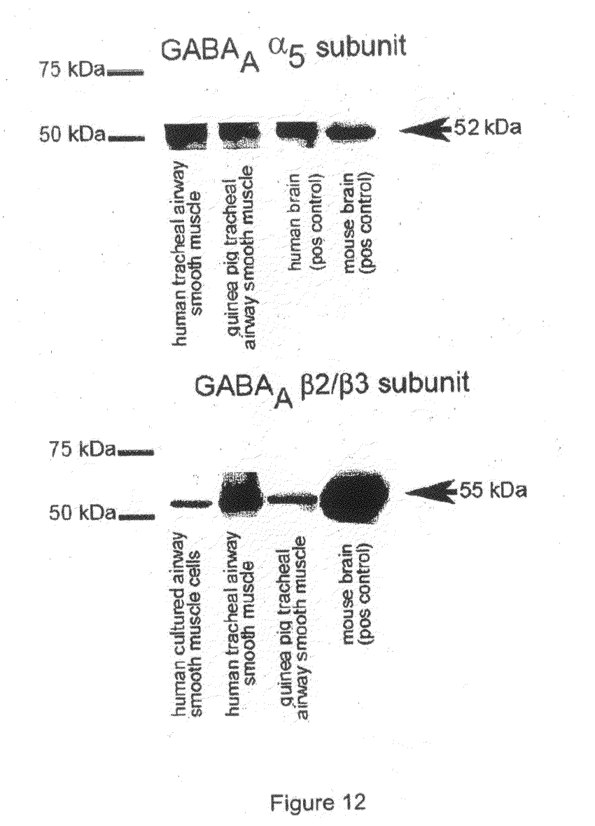

To explore these unexpected functional findings, the mRNA and protein expression of GABA_{A} subunits in native guinea pig and human airway smooth muscle, and cultured human airway smooth muscle cells were measured. mRNA encoding multiple subunits of GABA_{A} receptors was detected in airway smooth muscle cells from these three sources. (FIG. 11.) Lane 1 shows a negative control. Lane 2 shows RNA from cultured human airway smooth muscle cells. Lane 3 shows, RNA from native human airway smooth muscle. Lane 4 shows RNA from human whole brain (positive control). Additionally, immunoblot analysis identified selected GABA_{A} subunits in airway smooth muscle cells. (FIG. 12.) Despite careful dissection of native airway tissues, RNA extracted from these sources is not free from some RNA arising from other cell types (including neural). However, cultured airway smooth muscle cells are a homogenous population of smooth muscle without contaminating neural components.

### Example 10

### Expression of A β Subunit of GABA_{A} Receptors is Immunohistochemically Localized to Airway Smooth Muscle in Guinea Pig Tracheal Rings

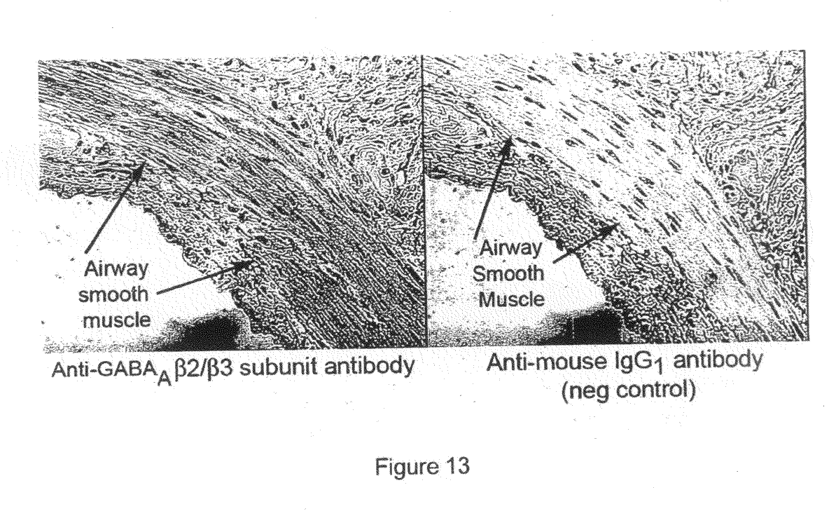

To further confirm the airway smooth muscle localization of GABA_{A} subunits, immunohistochemistry was performed in guinea pig airway rings. Abundant expression of one GABA_{A} subunit identified by RT-PCR and immunoblot is clearly localized to airway smooth muscle (brown staining). (FIG. 13.)

Having identified the expression of GABA_{A} receptors in airway smooth muscle, the source of the endogenous ligand for GABA_{A} receptors in airway smooth muscle was determined. Furthermore, the observation that propofol, a known allosteric potentiator of GABA action at GABA_{A} receptors, selectively relaxed an NK2 mediated contraction was investigated.

### Example 11

### Spontaneous Relaxation of an NK2 Mediated Contraction Occurs more Rapidly than an NK1-Mediated Contraction

One scenario that would account for the above findings is that activation of NK2 receptors in airways not only directly contracts airway smooth muscle but leads to the liberation of GABA from an airway neural source. The results presented in FIG. 10 are consistent with this hypothesis because a GABA_{A} antagonist blocked relaxation of a substance P contraction.

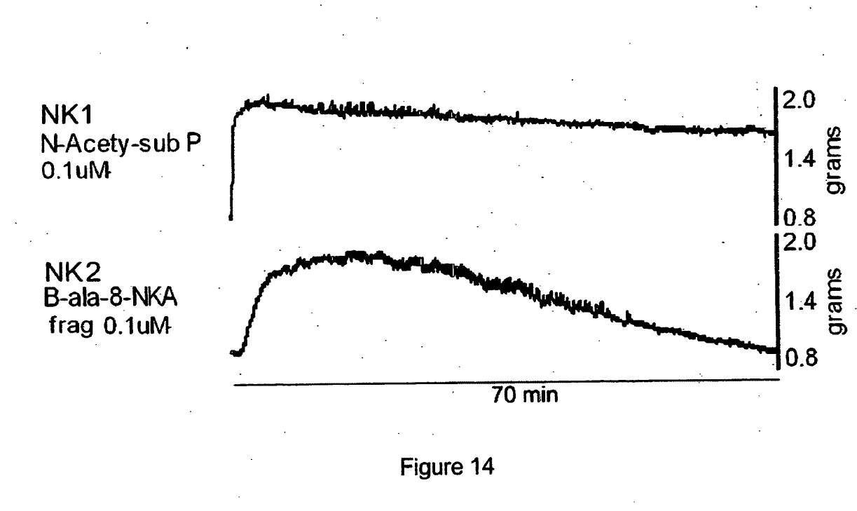

In this scenario, selective neural-induced release of GABA by an NK2 agonist would be expected to enhance the spontaneous relaxation of an NK2 mediated contraction due to the ability of liberated GABA to relax airway smooth muscle. An NK1 mediated contraction was sustained much longer than an NK2-mediated contraction consistent with the hypothesis that activation of NK2 receptors on neural structures induces the release of GABA. (FIG. 14.)

### Example 12

### Depletion of C Fibers or Blockade of C Fibers or Cholinergic Fibers Slowed the Spontaneous Relaxation of an NK2-Mediated Contraction

Possible neural sources of GABA in airways include cholinergic nerves and NANC nerves (C fibers). In vitro, the application of capsaicin to C fibers results in the irreversible depletion of tachykinins from C fibers. The sodium channel blocker teterodotoxin is known to block the activation of either C fibers or cholinergic nerves. Therefore, if GABA was being released from airway neural sources and was contributing to the spontaneous relaxation of an NK2-mediated contraction, then prior depletion or blockade of these neural sources should attenuate the spontaneous relaxation of an NK2-mediated contraction.

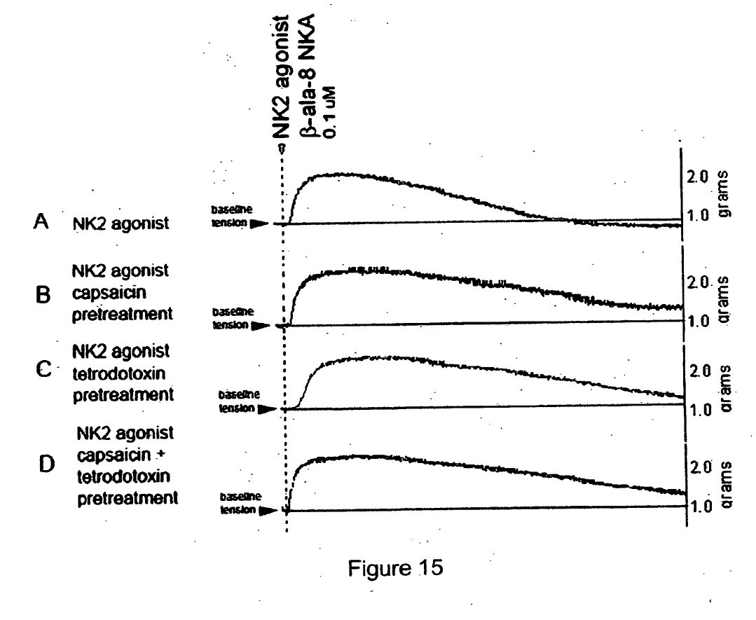

Consistent with the hypothesis that NK2-mediated liberation of GABA from an airway neural source was contributing to the relaxation of the contraction induced by activation of NK2 receptors on airway smooth muscle, depletion of C fibers or blockade of C fibers or cholinergic fibers slowed the relaxation of an NK2-mediated contraction. (FIG. 15.)

### Example 13

### Pretreatment of Guinea Pig Tracheal Rings with an Inhibitor of GABA Re-Uptake before the Application of Substance P Enhanced the Propofol-Mediated Relaxation of a Substance P-Induced Contraction

To further incriminate GABA as a contributor to propofol-mediated relaxation of a tachykinin-mediated contraction, guinea pig airway rings were pretreated with an inhibitor of GABA re-uptake (nipecotic acid 1 mM) and relaxation of substance-P induced contraction was measured. If GABA was being released and allowing for propofol's well known allosteric activity at GABA_{A} receptors (26-28), then an inhibitor of GABA re-uptake should further enhance relaxation.

Supporting the role of GABA in the phenomenon, pretreatment of guinea pig tracheal rings with an inhibitor of GABA re-uptake before the application of substance P enhanced the propofol-mediated relaxation of a substance P-induced contraction (FIG. 16.)

### Example 14

### Abundant Amounts of GABA were Immunohistochemically Detected In an Area Immediately Adjacent to Airway Smooth Muscle in Guinea Pig Tracheal Rings

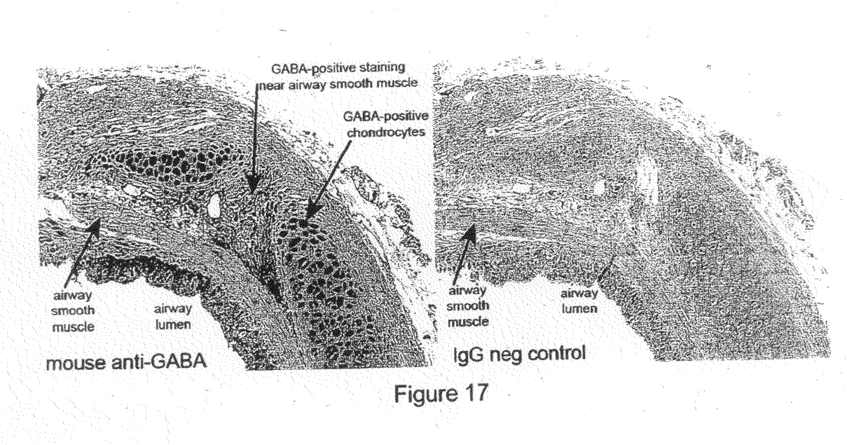

These multiple functional studies are consistent with a hypothesis that GABA released from an airway source acts on airway smooth muscle. Therefore, guinea pig airways were immunohistochemically stained for GABA. (FIG. 17.) Chondrocytes in cartilage are a known source of GABA and served as an internal staining control on the immunohistochemical study. FIG. 15 suggests that at least one source of GABA in the airway involves neural structures.

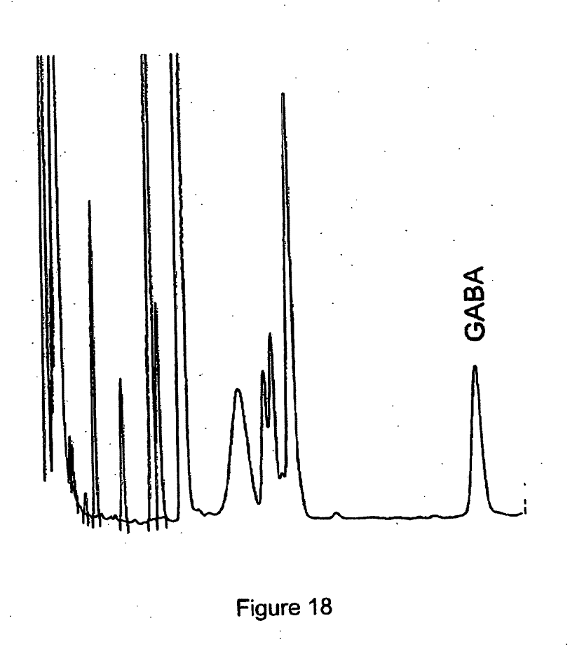

The measurement of liberated GABA from NK2-stimulated airways will further confirm these findings. FIG. 18 shows a chromatogram of amino acid neurotransmitters (including GABA) from a tissue lysate.

### Example 15

### Repetitive Tachykinin Airway Constrictions can be Induced in Guinea Pig Lungs In Vivo

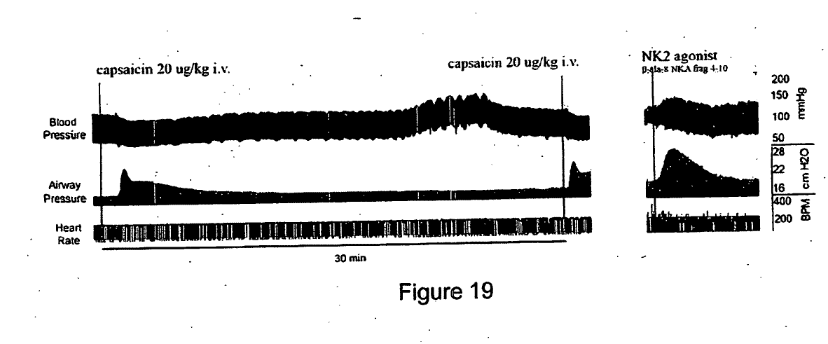

In vivo studies are carried out to confirm the in vitro findings. Guinea pigs are repetitively treated with intravenous neurokinin agonists or capsaicin to measure airway constriction induced by tachynergic events. FIG. 19 shows a study of airway pressure and hemodynamic measurements in an intact guinea pig treated with repetitive intravenous challenges of capsaicin resulting in similar changes in airway pressure.

One advantage of the in vivo airway model is that capsaicin-induced release of tachykinins does not result in irreversible loss of NANC neurotransmitters, these nerves are able to replenish their neurotransmitters for repetitive challenges. (FIG. 19.) FIG. 19 shows that airway pressure changes may be measured in response to an intravenous challenge with an NK2 receptor agonist. Further studies of this sort are carried out to confirm that repetitive tachykinin airway constrictions can be induced in guinea pig lungs in vivo.

### Example 16

### Human Airway Smooth Muscle Cells Express Functional GABA_{A} Receptors

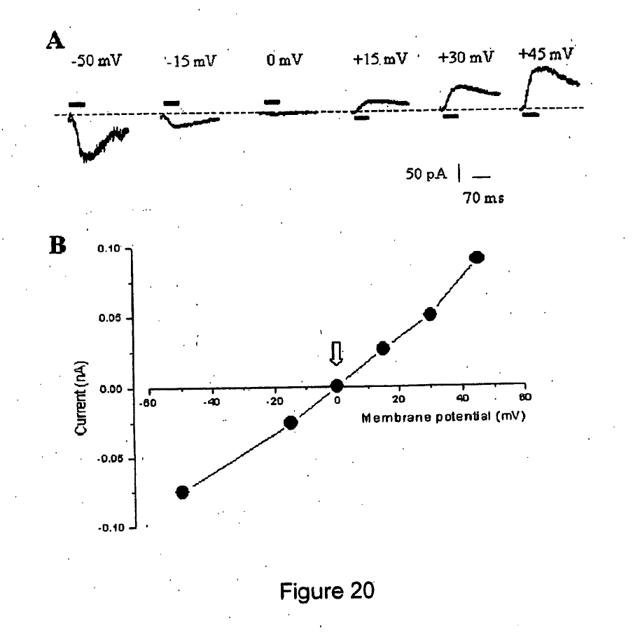

The effect of intravenous anesthetics on endogenously expressed GABA_{A} receptors in airway smooth muscle is measured. These electrophysiology studies require the measurement of chloride currents induced by GABA in airway smooth muscle cells. As seen in FIG. 20, a GABA-induced chloride current in airway smooth muscle cells has been measured, which further attests to the expression of GABA_{A} receptors in airway smooth muscle. Further studies of this sort are carried out to confirm that human airway smooth muscle cells express functional GABA_{A} receptors.

### Example 17

### HEK 293 Cells Transiently Transfected with GABA_{A} Subunits Express Functional Receptors Responsive to GABA

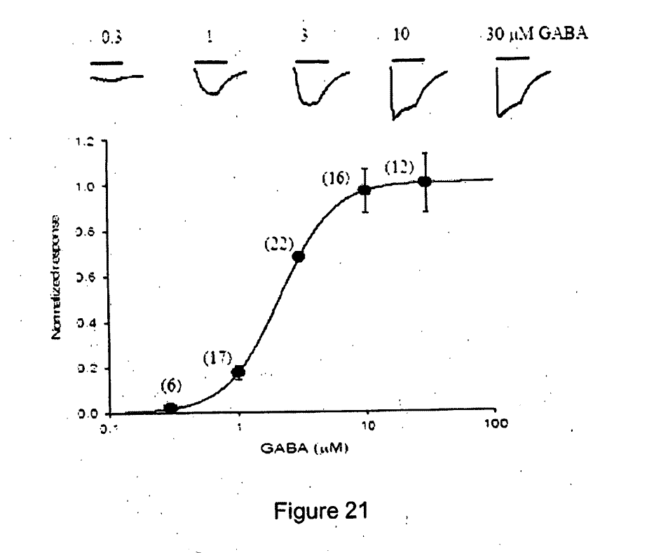

To demonstrate that the specific subunit combination dictates specificity of intravenous anesthetic effects at airway smooth muscle GABA_{A} receptors, GABA_{A} subunits identified in airway smooth muscle are transiently transfected into a heterologous expression system (HEK 293 cells). Functional GABA channels have been successfully transfected into HEK293 cells. (FIG. 21.) To demonstrate that the specific subunit composition of GABA_{A} receptors expressed in airway smooth muscle dictate their responsiveness to propofol as opposed to other intravenous anesthetics which are also known to allosterically enhance neural GABA_{A} channels, GABA_{A} subunits identified in guinea pig and human airway smooth muscle are recombinantly expressed in a hetertologous expression system and are evaluated for the interaction of propofol and other intravenous anesthetics with GABA_{A} receptors to illustrate the combinational specificity of GABA_{A} subunits to propofol responses in airway smooth muscle.

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents.

### BIBLIOGRAPHY

1. National Institutes of Health asthma incidence. Diseases and Conditions Index. http://www.nhlbi.nih.gov/health/dci/Diseases/Asthma/Asthma_WhoIsAtRisk.html
2. National Institutes of Health. COPD incidence. http://health.nih.gov/result.asp/165/15.
3. Pizov R, Brown R H, Weiss Y S, et al. Wheezing during induction of general anesthesia in patients with and without asthma: a randomized blinded trial. Anesthesiology 1995; 82:1111-6.
4. Eames W O, Rooke G A, Wu R S, Bishop M J. Comparison of the effects of etomidate, propofol, and thiopental on respiratory resistance after tracheal intubation. Anesthesiology 1996; 84:1307-11.
5. Yamakage M, Hirshman C A, Croxton T L. Inhibitory effects of thiopental, ketamine and propofol on voltage-dependent Ca²⁺ channels in porcine tracheal smooth muscle cells. Anesthesiology 1995; 83:1274-82.
6. Lin C C, Shyr M H, Tan P P, et al. Mechanisms underlying the inhibitory effect of propofol on the contraction of canine airway smooth muscle. Anesthesiology 1999; 91:750-9.
7. Cheng E Y, Mazzeo A J, Bosnjak Z J, et al. Direct relaxant effects of intravenous anesthetics on airway smooth muscle. Anesth Analg 1996; 83:162-8.
8. Ouedraogo N, Marthan R, Roux E. The effects of propofol and etomidate on airway contractility in chronically hypoxic rats. Anesth Analg 2003; 96:1035-41, table.
9. Hashiba E, Sato T, Hirota K, et al. The relaxant effect of propofol on guinea pig tracheal muscle is independent of airway epithelial function and beta-adrenoceptor activity. Anesth Analg 1999; 89:191-6.
10. Ouedraogo N, Roux E, Forestier F, et al. Effects of intravenous anesthetics on normal and passively sensitized human isolated airway smooth muscle. Anesthesiology 1998; 88:317-26.
11. Belouchi N E, Roux E, Savineau J P, Marthan R. Interaction of extracellular albumin and intravenous anaesthetics, etomidate and propofol, on calcium signalling in rat airway smooth muscle cells. Fundam Clin Pharmacol 2000; 14:395-400.
12. Hanazaki M, Jones K A, Warner D O. Effects of intravenous anesthetics on Ca2+ sensitivity in canine tracheal smooth muscle. Anesthesiology 2000; 92:133-9.
13. Kabara S, Hirota K, Hashiba E, et al. Comparison of relaxant effects of propofol on methacholine-induced bronchoconstriction in dogs with and without vagotomy. Br J Anaesth 2001; 86:249-53.
14. Hashiba E, Hirota K, Suzuki K, Matsuki A. Effects of propofol on bronchoconstriction and bradycardia induced by vagal nerve stimulation. Acta Anaesthesiol Scand 2003; 47:1059-63.
15. Brown R H, Wagner E M. Mechanisms of bronchoprotection by anesthetic induction agents: propofol versus ketamine. Anesthesiology 1999; 90:822-8.
16. Brown R H, Greenberg R S, Wagner E M. Efficacy of propofol to prevent bronchoconstriction: effects of preservative. Anesthesiology 2001; 94:851-5.
17. Dawidowicz A L, Kalitynski R, Kobielski M, Pieniadz J. Influence of propbfol concentration in human plasma on free fraction of the drug. Chem Biol Interact 2006; 159:149-55.
18. Fan S Z, Yu H Y, Chen Y L, Liu C C. Propofol concentration monitoring in plasma or whole blood by gas chromatography and high-performance liquid chromatography. Anesth Analg 1995; 81:175-8.
19. Dowrie R H, Ebling V V F, Mandema J W, Stanski D R. High-performance liquid chromatographic assay of propofol in human and rat plasma and fourteen rat tissues using electrochemical detection. J Chromatogr B Biomed Appl 1996; 678:279-88.
20. Conti G, Utri D D, Vilardi V, et al. Propofol induces bronchodilation in mechanically ventilated chronic obstructive pulmonary disease (COPD). patients. Acta Anaesthesiol Scand 1993; 37:105-9.
21. Hirota K, Ebina T, Sato T, et al. Is total body weight an appropriate predictor for propofol maintenance dose? Acta Anaesthesiol Scand 1999; 43:842-4.
22. Hoymork S C, Raeder J. Why do women wake up faster than men from propofol anaesthesia? Br J Anaesth 2005; 95:627-33.
23. Handa-Tsutsui F, Kodaka M. Propofol concentration requirement for laryngeal mask airway insertion was highest with the ProSeal, next highest with the Fastrach, and lowest with the Classic type, with target-controlled infusion. J Clin Anesth 2005; 17:344-7.
24. Luo W, Li Y H, Yang J J, et al. Cerebrospinal fluid and plasma propofol concentration during total intravenous anaesthesia of patients undergoing elective intracranial tumor removal. J Zhejiang Univ Sci B 2005; 6:865-8.
25. Kuipers J A, Boer F, Olieman W, et al. First-pass lung uptake and pulmonary clearance of propofol: assessment with a recirculatory indocyanine green pharmacokinetic model. Anesthesiology 1999; 91:1780-7.
26. Franks N P. Molecular targets underlying general anaesthesia. Br J Pharmacol 2006; 147 Suppl 1:S72-S81.
27. Concas A, Santoro G, Mascia M P, et al. The action of the general anesthetic propofol on GABAA receptors. Adv Biochem Psychopharmacol 1992; 47:349-63.
28. Trapani G, Altomare C, Liso G, et al. Propofol in anesthesia. Mechanism of action, structure-activity relationships, and drug delivery. Curr Med Chem 2000; 7:249-71.
29. Bormann J. The ‘ABC’ of GABA receptors. Trends Pharmacol Sci 2000; 21:16-9.
30. Simeone T A, Donevan S D, Rho J M. Molecular biology and ontogeny of gamma-aminobutyric acid (GABA) receptors in the mammalian central nervous system. J Child Neurol 2003; 18:39-48.
31. Stein V, Nicoll R A. GABA generates excitement. Neuron 2003; 37:375-8.
32. Kotlikoff M I, Kamm K E. Molecular mechanisms of beta-adrenergic relaxation of airway smooth muscle. Annu Rev Physiol 1996; 58:115-41.
33. Ghatta S, Nimmagadda D, Xu X, O'rourke S T. Large-conductance, calcium-activated potassium channels: Structural and functional implications. Pharmacol Ther 2005.
34. Salvail D, Dumoulin M, Rousseau E. Direct modulation of tracheal Cl-channel activity by 5,6- and 11,12-EET. Am J Physiol 1998; 275:L432-L441.
35. Jones-Davis D M, Macdonald R L. GABA(A) receptor function and pharmacology in epilepsy and status epilepticus. Curr Opin Pharmacol 2003; 3:12-8.
36. Mehta A K, Ticu M K. An update on GABAA receptors. Brain Res Brain Res Rev 1999; 29:196-217.
37. Benkwitz C, Banks M I, Pearce R A. Influence of GABAA receptor gamma2 splice variants on receptor kinetics and isoflurane modulation. Anesthesiology 2004; 101:924-36.
38. Li S F, Hu J H, Yan Y C, et al. Identification and characterization of a novel'splice variant of beta3 subunit of GABA(A) receptor in rat testis and spermatozoa. Int J Biochem Cell Biol 2005; 37:350-60.
39. Chang Y, Wang R, Barot S, Weiss D S. Stoichiometry of a recombinant GABAA receptor. J Neurosci 1996; 16:5415-24.
40. Baumann S W, Baur R, Sigel E. Forced subunit assembly in alpha1beta2gamma2 GABAA receptors. Insight into the absolute arrangement. J Biol Chem 2002; 277:46020-5.
41. Belelli D, Lambert J J. Neurosteroids: endogenous regulators of the GABA(A) receptor. Nat Rev Neurosci 2005; 6:565-75.
42. Carlton S M, Zhou S, Coggeshall R E. Peripheral GABA(A) receptors: evidence for peripheral primary afferent depolarization. Neuroscience 1999; 93:713-22.
43. Vassias I, Lecolle S, Vidal P P, de W C. Modulation of GABA receptor subunits in rat facial motoneurons after axotomy. Brain Res Mol Brain Res 2005; 135:260-75.
44. Calver A R, Medhurst A D, Robbins M J, et al. The expression of GABA(B1) and GABA(B2) receptor subunits in the cNS differs from that in peripheral tissues. Neuroscience 2000; 100:155-70.
45. Erdo S L. Identification of GABA receptor binding sites in rat and rabbit uterus. Biochem Biophys Res Commun 1984; 125:18-24.
46. Akinci M K, Schofield P R. Widespread expression of GABA(A) receptor subunits in peripheral tissues. Neurosci Res 1999; 35:145-53.
47. Ong J, Kerr D I. GABA-receptors in peripheral tissues. Life Sci 1990; 46:1489-501.
48. Taniguchi H, Okada Y, Seguchi H, et al. High concentration of gamma-aminobutyric acid in pancreatic beta cells. Diabetes 1979; 28:629-33.
49. Yang W, Reyes A A, Lan N C. Identification of the GABAA receptor subtype mRNA in human pancreatic tissue. FEBS Lett 1994; 346:257-62.
50. Racagni G, Apud I A, luliano E, et al. Anterior pituitary GABA receptors in relation to prolactin secretion. Adv Biochem Psychopharmacol 1983; 37:123-35.
51. Parramon M, Gonzalez M P, Oset-Gasque M J. Pharmacological modulation of adrenal medullary GABAA receptor: consistent with its subunit composition. Br J Pharmacol 1995; 116:1875-81.
52. Fujiwara M, Muramatsu I. Gamma-aminobutyric acid receptor on vascular smooth muscle of dog cerebral arteries. Br J Pharmacol 1975; 55:561-2.
53. Ferguson D R, Marchant J S. Inhibitory actions of GABA on rabbit urinary bladder muscle strips: mediation by potassium channels. Br J Pharmacol 1995; 115:81-3.
54. Amenta F, Cavallotti C, Ferrante F, Erdo S L. Autoradiographic visualization of the GABA-A receptor agonist, 3H-muscimol in the rat uterus. Pharmacol Res Commun 1988; 20:863-8.
55. Fujii E, Mellon S H. Regulation of uterine gamma-aminobutyric acid(A) receptor subunit expression throughout pregnancy. Endocrinology 2001; 142:1770-7.
56. Pencheva N, Venkova K, Radomirov R. GABAB receptor-mediated contractile effects resistant to tetrodotoxin in isolated cat ileum. Eur J Pharmacol 1990; 182:199-202.
57. Chen T F, Doyle P T, Ferguson D R. Inhibition in the human urinary bladder by gamma-amino-butyric acid. Br J Urol 1994; 73:250-5.
58. Pencheva N, Radomirov R. Biphasic GABA-A receptor-mediated effect on the spontaneous activity of the circular layer in cat terminal ileum. Gen Pharmacol 1993; 24:955-60.
59. Sbirakawa J, Nakanisbi T, Taniyama K, et al. Regulation of the substance P-induced contraction via the release of acetylcholine and gamma-aminobutyric acid in the guinea-pig urinary bladder. Br J Pharmacol 1989; 98:437-44.
60. Bayer S, Jellali A, Crenner F, et al. Functional evidence for a role of GABA receptors in modulating nerve activities of circular smooth muscle from rat colon in vitro. Life Sci 2003; 72:1481-93.
61. Napoleone P, Cavallotti C, de V G, Amenta F. Autoradiographic localization of the GABA-A-receptor agonist (3H)-muscimol in the rat intestinal musculature. Pharmacology 1991; 42:103-10.
62. Davies P A, McCartney M R, Wang W, et, al. Alternative transcripts of the GABA(A) receptor epsilon subunit in human and rat. Neuropharmacology 2002; 43:467-75.
63. Neelands T R, Macdonald R L. Incorporation of the pi subunit into functional gamma-aminobutyric Acid(A) receptors. Mol Pharmacol 1999; 56:598-610.
64. Hedblom E, Kirkness E F. A novel class of GABAA receptor subunit in tissues of the reproductive system. J Biol Chem 1997; 272: 15346-50.
65. Sarang S S, Plotkin M D, Gullans S R, et al. Identification of the gamma-aminobutyric acid receptor beta(2) and beta(3) subunits in rat, rabbit and human kidneys. J Am Soc Nephrol 2001; 12:1107-13.
66. Sun D, Gong Y, Kojima H, et al. Increasing cell membrane potential and GABAergic activity inhibits malignant hepatocyte growth. Am J Physiol Gastrointest Liver Physiol 2003; 285:G12-G19.
67. Erlitzki R, Gong Y, Zhang M, Minuk G. Identification of gamma-aminobutyric acid receptor subunit types in human and rat liver. Am J Physiol Gastrointest Liver Physiol 2000; 279:G733-G739.
68. Gorrie G H, Vallis Y, Stephenson A, et al. Assembly of GABAA receptors composed of alpha1 and beta2 subunits in both cultured neurons and fibroblasts. J Neurosci 1997; 17:6587-96.
69. Haxhiu M A, Deal E C, Jr., Norcia M P, et al. Medullary effects of nicotine and GABA on tracheal smooth muscle tone. Respir Physiol 1986; 64:351-63.
70. Moore C T, Wilson C G, Mayer C A, et al. A GABAergic inhibitory microcircuit controlling cholinergic outflow to the airways. J Appl Physiol 2004; 96:260-70.
71. Matera M G, D'Agostino B, Costantino M, et al. K+ channels and guinea-pig trachea: a possible functional modulation by GABAB receptors. Pulm Pharmacol 1994; 7:259-63.
72. Tohda Y, Ohkawa K, Kubo H, et al. Role of GABA receptors in the bronchial response: studies in sensitized guinea-pigs. Clin Exp Allergy 1998; 28:772-7.
73. Tamaoki J, Graf P D, Nadel J A. Effect of gamma-aminobutyric acid on neurally mediated contraction of guinea pig trachealis smooth muscle. J Pharmacol Exp Ther 1987; 243:86-90.
74. Chapman R W, Danko G, Rizzo C, et al. Prejunctional GABA-B inhibition of cholinergic, neurally-mediated airway contractions in guinea-pigs. Pulm Pharmacol 1991; 4:218-24.
75. Chapman R W, Danko G, del P M, et al. Further evidence for prejunctional GABA-B inhibition of cholinergic and peptidergic bronchoconstriction in guinea pigs: studies with new agonists and antagonists. Pharmacology 1993; 46:315-23.
76. Cirone J, Rosahl T W, Reynolds D S, et al. Gamma-aminobutyric acid type A receptor beta 2 subunit mediates the hypotbennic effect of etomidate in mice. Anesthesiology 2004; 100:1438-45.
77. Hill-Venning C, Belelli D, Peters J A, Lambert J J. Subunit-dependent interaction of the general anaesthetic etomidate with the gamma-aminobutyric acid type A receptor. Br J Pharmacol 1997; 120:749-56.
78. Belelli D, Lambert J J, Peters J A, et al. The interaction of the general anesthetic etomidate with the gamma-aminobutyric acid type A receptor is influenced by a single amino acid. Proc Natl Acad Sci USA 1997; 94:11031-6.
79. Reynolds D S, Rosahl T W, Cirone J, et al. Sedation and anesthesia mediated by distinct GABA(A) receptor isoforms. J Neurosci 2003; 23:8608-17.
80. Smith A J, Oxley B, Malpas S, et al. Compounds exhibiting selective efficacy for different beta subunits of human recombinant gamma-aminobutyric acid A receptors. J Pharmacol Exp Ther 2004; 311:601-9.
81. Ranna M, Sinkkonen S T, Moykkynen T, et al. Impact of epsilon and theta subunits on pharmacological properties of alpha3beta1 GABAA receptors expressed in Xenopus oocytes. BMC Pharmacol 2006; 6:1.
82. Coleridge J C, Coleridge H M. Afferent vagal C fibre innervation of the lungs and airways and its functional significance. Rev Physiol Biochem Pharmacol 1984; 99:1-110.
83. Kummer W, Fischer A, Kurkowski R, Heym C. The sensory and sympathetic innervation of guinea-pig lung and trachea as studied by retrograde neuronal tracing and double-labelling immunohistochemistry. Neuroscience 1992; 49:715-37.
84. Ricco M M, Kummer W, Biglari B, et al. Interganglionic segregation of distinct vagal afferent fibre phenotypes in guinea-pig airways. J Physiol 1996; 496 (Pt 2):521-30.
85. Solway J, Leff A R. Sensory neuropeptides and airway function. J Appl Physiol 1991; 71:2077-87.
86. Coleridge H M, Coleridge J C, Schultz H D. Afferent pathways involved in reflex regulation of airway smooth muscle. Pharmacol Ther 1989; 42:1-63.
87. Fuller R W, Dixon C M, Cuss F M, Barnes P J. Bradykinin-induced bronchoconstriction in humans. Mode of action. Am Rev Respir Dis 1987; 135:176-80.
88. Lee L Y, Pisarri T E. Afferent properties and reflex functions of bronchopulmonary C-fibers. Respir Physiol 2001; 125:47-65.
89. Sant'Ambrogio G, Widdicombe J. Reflexes from airway rapidly adapting receptors. Respir Physiol 2001; 125:33-45.
90. Sheppard D, Epstein J, Skoogh B E, et al. Variable inhibition of histamine-induced bronchoconstriction by atropine in subjects with asthma. J Allergy Clin Immunol 1984; 73:82-7.
91. Rizzo C A, Valentine A F, Egan R W, et al. NK(2)receptor mediated contraction in monkey, guinea-pig and human airway smooth muscle. Neuropeptides 1999; 33:27-34.
92. Venugopal C S, Christopher C L, Wilson S M, et al. Pharmacologic evaluation of neurokinin-2 receptor antagonists in the guinea pig respiratory tract. Am J Vet Res 2004; 65:984-91.
93. Mukaiyama O, Morimoto K, Nosaka E, et al. Involvement of enhanced neurokinin NK3 receptor expression in the severe asthma guinea pig model. Eur J Pharmacol 2004; 498:287-94.
94. Barnes P J. Neuropeptides and asthma. Am Rev Respir Dis 1991; 143:S28-S32.
95. Ichinose M, Nakajima N, Takahashi T, et al. Protection against bradykinin-induced bronchoconstriction in asthmatic patients by neurokinin receptor antagonist. Lancet 1992; 340:1248-51.
96. Joos G F, De Swert K O, Pauwels R A. Airway inflammation and tachykinins: prospects for the development of tachykinin receptor antagonists. Eur J Pharmacol 2001; 429:239-50.
97. Joos G F, De Swert K O, Schelfhout V, Pauwels R A. The role of neural inflammation in asthma and chronic obstructive pulmonary disease. Ann N Y Acad Sci 2003; 992:218-30.
98. Cohen J, Burggraaf J, Schoemaker R C, et al. Relationship between airway responsiveness to neurokinin A and methacholine in asthma. Pulm Pharmacol Ther 2005; 18:171-6.
99. Joos G F, Vincken W, Louis R, et al. Dual tachykinin NK1/NK2 antagonist DNK333 inhibits neurokinin A-induced bronchoconstriction in asthma patients. Eur Respir J 2004; 23:76-81.
100. Schelfout V, Louis R, Lenz W, et al. The triple neurokinin-receptor antagonist CS-003 inhibits neurokinin A-induced bronchoconstriction in patients with asthma. Pulm Pharmacol Ther 2005.
101. Lecci A, Giuliani S, Tramontana M, et al. Peripheral actions of tachykinins. Neuropeptides 2000; 34:303-13.
102. Willis W D. Role of neurotransmitters in sensitization of pain responses. Ann NY Acad Sci 2001; 933:142-56.
103. Urban L, Thompson S W, Dray A. Modulation of spinal excitability: co-operation between neurokinin and excitatory amino acid neurotransmitters. Trends Neurosci 1994; 17:432-8.
104. Chuaychoo B, Hunter D D, Myers A C, et al. Allergen-induced substance P synthesis in large-diameter sensory neurons innervating the lungs. J Allergy Clin Immunol 2005; 116:325-31.
105. Myers A C, Undem B J. Electrophysiological effects of tachykinins and capsaicin on guinea-pig bronchial parasympathetic ganglion neurones. J Physiol 1993; 470:665-79.
106. Canning B J, Reynolds S M, Anukwu L U, et al. Endogenous neurokinins facilitate synaptic transmission in guinea pig airway parasympathetic ganglia. Am J Physiol Regul Integr Comp Physiol 2002; 283:R320-R330.
107. Myers A C, Goldie R G, Hay D W. A novel role for tachykinin neurokinin-3 receptors in regulation of human bronchial Ganglia neurons. Am J Respir Crit Care Med 2005; 171:212-6.
108. Zhang X L, Mak J C, Bames P J, Characterization and autoradiographic mapping of [3H]CP96,345, a nonpeptide selective NK1 receptor antagonist in guinea pig lung. Peptides 1995; 16:867-72.
109. Zhang G, Wang L, Liu H, Zhang J. Substance P promotes sleep in the ventrolateral preoptic area of rats. Brain Res 2004; 1028:225-32.
110. Stacey A E, Woodhall G L, Jones R S. Activation of neurokinin-1 receptors promotes GABA release at synapses in the rat entorhinal cortex. Neuroscience 2002; 115:575-86,
111. Preston Z, Richardson P J, Pinnock R D, Lee K. NK-3 receptors are expressed on mouse striatal gamma-aminobutyric acid-ergic interneurones and evoke [(3)H] gamma-aminobutyric acid release. Neurosci Lett 2000; 284:89-92.
112. Brouillette J, Couture R. Evidence for a GABA(B) receptor component in the spinal action of Substance P (SP) on arterial blood pressure in the awake rat. Br J Pharmacol 2002; 136:1169-77.
113. Maehara T, Suzuki H, Yoshioka K, Otsuka M. Characteristics of substance P-evoked release of amino acids from neonatal rat spinal cord. Neuroscience 1995; 68:577-84.
114. Stanski D R, Maitre P O. Population pharmacokinetics and pharmacodynamics of thiopental: the effect of age revisited. Anesthesiology 1990; 72:412-22.
115. Cordato D J, Mather L E, Gross A S, Herkes G K. Pharmacokinetics of thiopental enantiomers during and following prolonged high-dose therapy. Anesthesiology 1999; 91:1693-702.
116. Wieber J, Gugler R, Hengstmann J H, Dengler H J. Pharmacokinetics of ketamine in man. Anaesthesist 1975; 24:260-3.
117. Van Hamme M J, Ghoneim M M, Ambre J J. Pharmacokinetics of etomidate, a new intravenous anesthetic. Anesthesiology 1978; 49:274-7.

What is claimed is:

1. A method of mitigating bronchospasm or airway smooth muscle constriction due to irritation comprising administering to a subject in need of such treatment an amount of propofol or a derivative thereof effective to decrease the severity and/or duration of bronchospasm or airway smooth muscle constriction.

2. A method according to claim 1, comprising administering to a subject in need of such treatment an amount of propofol effective to decrease the severity and/or duration of bronchospasm or airway smooth muscle constriction.

3. A method according to claim 1, wherein the effective amount is from about 1% (wt) to about 15% (wt).

4. A method according to claim 3, wherein the effective amount is from about 2% (wt) to about 10% (wt).

5. A method according to claim 4, wherein the effective amount is from about 3% (wt) to about 5% (wt).

6. A method according to claim 1, wherein the propofol or a derivative thereof is administered by inhalation.

7. A method according to claim 1, wherein the propofol or a derivative thereof is administered in the form of a pharmaceutical formulation comprising the propofol or a derivative thereof and a pharmaceutically acceptable carrier.

8. A method of up-regulating GABA mediated relaxation of airway smooth muscle at GABA_{A} receptors expressed on airway smooth muscle comprising administering to a subject an amount of propofol or a derivative thereof effective to increase the speed of the spontaneous relaxation of the airway smooth muscle.

9. A method according to claim 8, comprising administering to a subject an amount of propofol effective to increase the speed of the spontaneous relaxation of the airway smooth muscle.

10. A method according to claim 8, wherein the effective amount is from about 1% (wt) to about 15% (wt).

11. A method according to claim 10, wherein the effective amount is from about 2% (wt) to about 10% (wt).

12. A method according to claim 11, wherein the effective amount is from about 3% (wt) to about 5% (wt).

13. A method according to claim 8, wherein the propofol or a derivative thereof is administered by inhalation.

14. A method according to claim 8, wherein the propofol or a derivative thereof is administered in the form of a pharmaceutical formulation comprising the propofol or a derivative thereof and a pharmaceutically acceptable carrier.

15. A method of anesthetizing a subject and minimizing bronchospasm or airway smooth muscle constriction due to irritation comprising administering an effective amount of propofol or a derivative thereof directly to the airway smooth muscle concurrently with anesthetization.

16. A method according to claim 15, comprising administering an effective amount of propofol directly to the airway smooth muscle concurrently with anesthetization.

17. A method according to claim 15, wherein the effective amount is from about 1% (wt) to about 15% (wt).

18. A method according to claim 17, wherein the effective amount is from about 2% (wt) to about 10% (wt).

19. A method according to claim 18, wherein the effective amount is from about 3% (wt) to about 5% (wt).

20. A method according to claim 15, wherein the propofol or a derivative thereof is administered by inhalation.
